(19) United States
(12) Patent Application Publication
(10) Pub. No.: US 2023/0277827 A1
(43) Pub. Date: Sep. 7, 2023

(54) RECOMBINANT SUBUNIT VACCINE MICRONEEDLE PRODUCT AS WELL AS PREPARATION METHOD AND APPLICATION THEREOF

(71) Applicants: Healthina Stem Cell Industry Platform (Tianjin) Limited, Tianjin (CN); Tangyi Holdings(Shenzhen) Limited, Shenzhen (CN)

(72) Inventors: Bin ZHENG, Tianjin (CN); Yulin CAO, Beijing (CN); Wei SUN, Tianjin (CN); Shixiang CHENG, Tianjin (CN)

(73) Assignees: Healthina Stem Cell Industry Platform (Tianjin) Limited, Tianjin (CN); Tangyi Holdings(Shenzhen) Limited, Shenzhen (CN)

(21) Appl. No.: 17/751,695

(22) Filed: May 24, 2022

(30) Foreign Application Priority Data
Mar. 2, 2022 (CN) 202210197942.8

(51) Int. Cl. A61M 37/00; A61K 39/12; A61K 9/00; A61K 47/38

(52) U.S. Cl. CPC A61M 37/0015; A61K 39/12; A61K 9/0021; A61K 47/38; A61M 2037/0023; A61M 2037/0046; A61M 2037/0061

(57) ABSTRACT

A recombinant subunit vaccine microneedle product includes a backing and a recombinant subunit lung-associated virus vaccine solution-containing microneedle array attached to a side of the backing, wherein the recombinant subunit lung-associated virus vaccine solution-containing microneedle array includes a plurality of microneedles, wherein each microneedle contains a matrix and a recombinant subunit loaded in the matrix. The present invention adopts the recombinant subunit vaccine microneedle product as well as a preparation method and an application thereof, wherein such microneedle product can realize a rapid humoral immunity after being administered to the skins of animal bodies (especially human bodies), which solves the problems of traditional lung-associated virus vaccine injection, such as muscular pain and multiple injections.

### CROSS REFERENCE TO THE RELATED APPLICATIONS

This application is based upon and claims priority to Chinese Patent Application No. 202210197942.8, filed on Mar. 2, 2022, the entire contents of which are incorporated herein by reference.

### TECHNICAL FIELD

The present invention relates to the technical field of biological microneedles, specifically relates to a recombinant subunit lung-associated virus vaccine microneedle product in which microneedles are included to perform lung-associated virus vaccine injection, in particular to a recombinant subunit vaccine microneedle product as well as a preparation method and an application thereof.

### BACKGROUND

The epidemic around the world is still very serious, and has resulted in immeasurable losses to the economy and people's lives of all countries. As the epidemic is gradually controlled, widespread inoculation of lung-associated virus vaccines plays a relatively important role. Current lung-associated virus vaccines for pneumonia include inactivated lung-associated virus vaccines, recombinant subunit lung-associated virus vaccines, adenovirus vector lung-associated virus vaccines and nucleic acid lung-associated virus vaccines, which are mainly administrated by intramuscular injection.

However, the intramuscular injection presents some insurmountable problems. For example, cold chain storage is required, and cold chain storage greatly increases the costs of transportation, storage and use; the intramuscular injection requires a certain injection dose to take effect, generally one or two or more injections are required, and both the use of medical resources and the time arrangement of vaccines are problematic.

Served as a new technique of locally transdermal administration, microneedle administration combines the convenience of emplastrum and the effectiveness of subcutaneous injection, avoids the shortcomings of other administration methods, and features the advantages of no nerve access, safety, no pain, efficient penetration, etc. However, it is difficult to store a lung-associated virus vaccine in microneedle patches in the prior art.

### SUMMARY

The present invention is intended to provide a recombinant subunit vaccine microneedle product as well as a preparation method and an application thereof, wherein such microneedle product realizes a rapid immune response by loading a recombinant subunit lung-associated virus vaccine-containing solution in the microneedle product after being administered to the skin, and a long-acting stable release and rapid response of a lung-associated virus vaccine are achieved.

To achieve the aforesaid purposes, the present invention provides a recombinant subunit vaccine microneedle product, including a backing and a recombinant subunit lung-associated virus vaccine solution-containing microneedle array attached to a side of the backing, wherein the recombinant subunit lung-associated virus vaccine solution-containing microneedle array includes a plurality of microneedles, wherein each microneedle contains a matrix and a recombinant subunit loaded in the matrix.

Preferably, the recombinant subunit lung-associated virus vaccine is obtained by extracting a special protein structure of bacteria and viruses through a chemical decomposition or controlled proteolysis method, and screening out a fragment lung-associated virus vaccine with immunocompetence.

Preferably, the matrixes are formed by crosslinking and/or drying and curing of an aqueous solution containing one or more of the following substances: polyethylene glycol diacrylate, silk fibroin, methacrylate gelatin (GelMA), carboxymethyl cellulose, trehalose, hyaluronic acid, polylactic acid-glycolic acid copolymer, polylactic acid, galactose, polyvinylpyrrolidone (PVP) or polyvinyl alcohol, preferably GelMA and/or hyaluronic acid, and more preferably GelMA.

Preferably, the backing is formed by crosslinking and/or drying and curing of an aqueous solution containing one or more of the following substances: polyethylene glycol diacrylate, silk fibroin, GelMA, carboxymethyl cellulose, trehalose, hyaluronic acid, polylactic acid-glycolic acid copolymer, polylactic acid, galactose, PVP or polyvinyl alcohol, and preferably hyaluronic acid.

Preferably, each microneedle includes a tip and a bottom, wherein the tip is away from the backing, each microneedle is attached to the backing via the bottom, a distance from the tip to the bottom is 200 μm-1 mm, a diameter of the bottom is 100 μm-500 μm, and a spacing between the adjacent tips is 300 μm-800 μm.

A method for preparing the recombinant subunit vaccine microneedle product, including the following steps:
(1) preparing a recombinant subunit lung-associated virus vaccine solution: extracting a special protein structure of bacteria and viruses through a chemical decomposition or controlled proteolysis method, and preparing screened fragments with immunocompetence into a lung-associated virus vaccine solution;
(2) adding a matrix material forming a matrix to the recombinant subunit-containing liquid medium obtained in step (1) to form a mixed solution; wherein in the mixed solution, the matrix material accounts for 20%-40% of a total weight of the mixed solution, preferably 25%-35%, and more preferably 30%;
(3) preparing a microneedle mold;
(4) placing the mixed solution obtained in step (2) in molding holes of the microneedle mold, and filling at least a portion of the volume of the molding holes, and preferably the whole volume of the molding holes;
(5) crosslinking the mixed solution containing the matrix material and the recombinant subunit in the molding holes, and preferably performing ultraviolet crosslinking for 5 s-15 s, and preferably 10 s, and/or performing drying and curing, and preferably performing hot curing, to form microneedles in the molding holes; wherein a plurality of microneedles form a recombinant subunit lung-associated virus vaccine microneedle array, and each microneedle includes a tip and a bottom, and the tip is away from an upper surface relative to the bottom;
(6) applying a backing material-containing solution to bottom surfaces of the microneedles and an upper surface of the microneedle mold that is not covered by the microneedles to form a backing solution layer, and crosslinking the backing material to form a continuous backing layer, so that the recombinant subunit lung-associated virus vaccine microneedle array is attached to the backing solution layer or the backing layer; and
(7) drying and curing the backing solution layer or the backing layer obtained in step (6) and the recombinant subunit lung-associated virus vaccine microneedle array to form a subunit vaccine microneedle product.

Preferably, the microneedle mold includes an upper surface and molding holes extending downward from the upper surface, wherein each of the molding holes includes a tip and a bottom, the tip is away from the upper surface, the bottom plane is flush with the upper surface, a distance from the tip to the bottom is 200 μm-1 mm, a diameter of the bottom is 100 μm-500 μm, and a spacing between the adjacent tips is 300 μm-800 μm.

The present invention further provides an application of the recombinant subunit vaccine microneedle product in preparing medical devices or drugs against pneumonia.

Therefore, the present invention adopts the recombinant subunit vaccine microneedle product as well as a preparation method and an application thereof. With full use of the advantages of the microneedle product and the recombinant subunit lung-associated virus vaccine, an immune response can be caused through certain components derived from pathogenic bacteria or viruses, i.e., highly purified proteins or synthetic peptides. The lung-associated virus vaccine present in the microneedle patch can not only work for a long time, but also reduce the discomfort caused by intramuscular injection and the use of medical resources.

A microneedle product generally includes a plurality of microneedles with a length of no more than 1 mm. The microneedles can form micro channels in the skin cuticle, break through the barrier of the skin cuticle and promote the penetration of drugs, thus reducing a dose of drugs accumulated in the cuticle, increasing a dose of drugs reaching the epidermal, dermal and subcutaneous tissues, and promoting the transdermal absorption of small-molecule and large-molecule drugs.

The present invention features the following specific technical effects:

(1) According to the subunit vaccine microneedle product of the present invention, a recombinant protein is produced through engineered cells. The production process is stable and reliable, can quickly realize a large-scale industrial production at home and abroad, and significantly reduces a production cost of the lung-associated virus vaccine, moreover, the storage and transportation are convenient.

(2) The subunit lung-associated virus vaccine of the present invention just has a few major surface proteins, avoiding generation of multiple unrelated antigen-induced antibodies, thus reducing side effects and relevant diseases as a result of the lung-associated virus vaccine.

(3) The subunit vaccine microneedle product of the present invention can pierce the skin cuticle which limits drug absorption, and promote the spread of recombinant subunit proteins into a human body without causing pain.

(4) Microneedles are prepared by a microneedle template reverse mold. The method is simple, easy to operate, low-cost, reusable, easy to control the basic shape of a microneedle array, highly safe, and suitable for promotion, without needing high technical requirements.

The technical solutions of the present invention will be further described below in detail in combination with the accompanying drawings and embodiments.

### BRIEF DESCRIPTION OF THE DRAWINGS

FIG. 1 is a structural side view of a part of a recombinant subunit vaccine microneedle product.

FIG. 2 is a side view of a microneedle mold of a recombinant subunit vaccine microneedle product.

### LABELS IN THE FIGURES

100. recombinant subunit vaccine microneedle product; 110. microneedle; 120. backing; 200. microneedle mold; 201. molding hole; 202. upper surface.

### DETAILED DESCRIPTION OF THE EMBODIMENTS

The technical solutions of the present invention will be further described below in combination with the accompanying drawings and embodiments.

Unless otherwise defined, the technical or scientific terms used herein should have ordinary meanings understood by those of ordinary skill in the art of the present invention.

It is apparent to those skilled in the art that the present invention is not limited to the details of the above-mentioned exemplary embodiments and can be realized in other specific forms without departing from the intention or essential features of the present invention. Therefore, in all respects, the embodiments should be considered to be exemplary and non-restrictive. The scope of the present invention is limited by the appended claims rather than the above-mentioned description, so that all changes falling within the meaning and scope of the equivalents of the claims are intended to be included in the present invention, and any accompanying drawing marks in the claims should not be deemed to limit the claims involved.

Moreover, it should be understood that although the specification is described according to the implementation modes, not each implementation mode contains only one independent technical solution. This narrative form of the specification is for the sake of clarity only. Those skilled in the art should take the specification as a whole, and the technical solutions in various embodiments may be combined appropriately to form other implementation modes that can be understood by those skilled in the art. These other implementation modes should also fall within the protection scope of the present invention.

The above-mentioned specific embodiments should also be understood as only for explaining the present invention, and the protection scope of the present invention is not limited to this. Within the technical scope disclosed by the present invention, the equivalent substitutions or changes made by those of skilled in the art based on the technical solutions and inventive concept of the present invention should fall within the protection scope of the present invention/invention.

The “including/comprising” or “containing” and similar words used herein refer to that the element ahead of the word covers the elements listed behind the word and does not exclude the possibility of covering other elements as well. The orientations or position relations indicated by terms “inside”, “outside”, “up” and “down” are those shown based on the accompanying drawings, only used for the convenience of describing the present invention and simplifying the description, rather than indicating or implying that the device or element referred to must have a particular orientation or be constructed and operated in a particular orientation, so they cannot be understood as a limitation to the present invention. When the absolute position of the object described changes, the relative position relation may also change accordingly. In the present invention, unless otherwise expressly specified and limited, the term “attaching” should be understood in a broad sense. For example, two elements may be connected fixedly, connected detachably, or integrated; two elements may be connected directly, or connected indirectly through an intermediate medium, or communicated internally or interact. Those of ordinary skill in the art can understand the specific meanings of such terms in the present invention according to the specific situations. The term “about” used herein has the meaning known to those skilled in the art, and preferably refers to that the value modified by the term is within the range of ±50%, ±40%, ±30%, ±20%, ±10%, ±5% or ±1%.

All terms (including technical or scientific terms) used in the disclosure have the same meanings as those understood by those of ordinary skill in the art of the disclosure, unless otherwise specifically defined. Moreover, it should be understood that terms defined in a general dictionary should be understood to have meanings consistent with those in the context of the relevant techniques, and should not be interpreted in an idealized or highly formal sense, unless expressly defined herein.

The techniques, methods and equipment known to those of ordinary skill in the art may not be discussed in detail, but where appropriate, such techniques, methods and equipment should be considered as a part of the specification.

The contents disclosed in the prior art literature referenced in the specification of the present invention are incorporated herein by reference in its entirety.

### Example 1

The recombinant subunit vaccine microneedle product 100 includes a backing and a recombinant subunit lung-associated virus vaccine microneedle array attached to a side of the backing 120, wherein the recombinant subunit lung-associated virus vaccine microneedle array includes a plurality of microneedles 110, wherein each microneedle 110 contains a matrix and a recombinant subunit lung-associated virus vaccine solution loaded in the matrix.

In the subunit vaccine microneedle product 100, there is no special limitation to a matrix material forming a matrix, and all common matrix materials for preparing the microneedle product adopted in the field can be applied in the present invention. However, considering that the microneedles 110 formed in the present invention need to have a certain mechanical strength, and the cured microneedles 110 need to have a certain porosity, the matrixes are formed by crosslinking and/or drying and curing of an aqueous solution containing one or more of the following substances: polyethylene glycol diacrylate, silk fibroin, GelMA, carboxymethyl cellulose, trehalose, hyaluronic acid, polylactic acid-glycolic acid copolymer, polylactic acid, galactose, PVP or polyvinyl alcohol, preferably GelMA and/or hyaluronic acid, and more preferably GelMA.

Further, the matrix material is preferably GelMA and/or hyaluronic acid. The GelMA is prepared from methacrylic anhydride (MA) and gelatin, which are crosslinked by UV light under the mediation of photosensitizer to form a pore structure with a certain strength. The GelMA features excellent biocompatibility. The hyaluronic acid is a mucopolysaccharide, which has a function of skin protection and can be applied to accelerate wound healing.

In the subunit vaccine microneedle product 100, there is no special limitation to a backing material forming a backing 120, and all common backing materials for preparing the microneedle product adopted in the field can be applied in the present invention. However, in the present invention, considering that the formed backing 120 needs to have a certain mechanical strength and flexibility, the backing 120 is preferably formed by crosslinking and/or drying of an aqueous solution containing one or more of the following substances: polyethylene glycol diacrylate, silk fibroin, GelMA, carboxymethyl cellulose, trehalose, hyaluronic acid, polylactic acid-glycolic acid copolymer, polylactic acid, galactose, PVP or polyvinyl alcohol, and preferably hyaluronic acid.

Further, the hyaluronic acid is a mucopolysaccharide, which has a function of skin protection and can be applied to accelerate wound healing, so such hyaluronic acid is preferably used as a backing material in the present invention.

Herein, there is no limitation to a thickness of the formed backing 120, but considering that the backing 120 needs to have a certain strength and flexibility, such thickness is preferably 0.1-15 mm, more preferably 1-10 mm, and most preferably 2-3 mm.

Further, in some cases, in the subunit vaccine microneedle product 100, materials forming the matrixes and the backing 120 are the same. In these cases, the microneedles 110 and the backing 120 are combined more stably, and in the following method for preparing the subunit vaccine microneedle product 100, the microneedle array and the backing 120 can be integrated, simplifying a preparation process. In other cases, the materials of the matrixes and the backing 120 are different, e.g., the matrix material is GelMA, while the backing material is hyaluronic acid. In such cases, the microneedles 110 have a certain strength, a pore structure and excellent biocompatibility; in addition, the backing 120 has a better protective effect on the skin, and can be applied to accelerate wound healing.

It should be noted that those skilled in the art are fully capable of appropriately selecting matrix and backing materials according to the desired application.

In addition, in the subunit vaccine microneedle product 100, there is no special limitation to the size and shape of the microneedles 110, and both can vary over a wide range according to an application part of the subunit vaccine microneedle product 100.

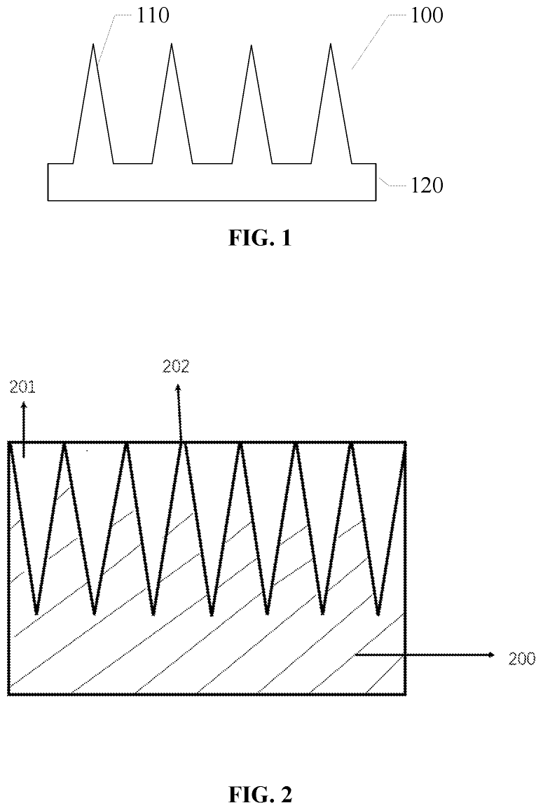

As shown in FIG. 1, in the subunit vaccine microneedle product 100, each microneedle 110 includes a tip and a bottom, wherein the tip is away from the backing 120, each microneedle 110 is attached to the backing 120 via the bottom, and there is no special limitation to a height between the tip and the bottom, but preferably 200 μm-1 mm. The height is no lower than 200 μm, otherwise the microneedles 110 will not pierce some animal bodies, preferably the skin cuticles of some parts of a human body. However, the height is no higher than 1 mm, otherwise the microneedles will pierce some animal bodies, preferably the skin cuticles of some parts of a human body, and reach the nervous layer, thus causing pain.

As shown in FIG. 1, in the subunit vaccine microneedle product 100, the bottom of each of the microneedles 110 has a diameter of 100 μm-500 μm. The diameter of the bottom is preferably no less than 100 μm, otherwise the microneedles 110 will be easy to break due to insufficient mechanical strength. In addition, the diameter of the bottom is no more than 500 μm, otherwise after the subunit vaccine microneedle product 100 is applied to some animal bodies, preferably some parts of a human body, larger holes will be left on the skin, leading to problems concerning skin beauty and healing.

In addition, in the subunit vaccine microneedle product 100, there is no special limitation to a stereo shape of the microneedles 110, and the microneedles can be in a shape of cylinder, cone, circular truncated cone, etc., or a combination of them, and preferably regular or irregular circular cone, conoid, triangular pyramid, rectangular pyramid or higher-level pyramid which may be a right cone or an oblique cone.

As shown in FIG. 1, in the subunit vaccine microneedle product 100, a spacing between the adjacent tips of the microneedles 110 is 300 μm-800 μm. The spacing within such range can make the subunit vaccine microneedle product 100 achieve an optimization effect in a depth of piercing the cuticle, etc.

It should be noted that those skilled in the art are capable of appropriately selecting shapes, sizes, etc. of the microneedles 110 in the subunit vaccine microneedle product 100 according to practical application situations.

### Example 2

A method for preparing the recombinant subunit vaccine microneedle product 100 includes the following steps:

(1) A recombinant subunit lung-associated virus vaccine solution was prepared: In a variety of specific antigenic determinants carried by macromolecule antigens, only a small number of antigenic sites contributed to a protective immune response. A special protein structure of bacteria and viruses was extracted through a chemical decomposition or controlled proteolysis method, and screened fragments with immunocompetence were prepared into a lung-associated virus vaccine solution.

(2) A matrix material forming a matrix was added to the recombinant subunit lung-associated virus vaccine-containing solution obtained in step (1) to form a mixed solution.

In step (2), there is no special limitation to a concentration of the matrix material in the mixed solution, as long as an amount of the matrix material is sufficient to form microneedles 110 in a final recombinant subunit lung-associated virus vaccine microneedle product 100 of the present invention. However, the matrix material in the liquid medium is 20%-40% (by weight), preferably 25%-35%, and more preferably 30%. In such ranges, the microneedles 110 formed by the matrix material have sufficient mechanical strength and a certain porosity to achieve an optimum hydrogen generation efficiency.

(3) A microneedle mold 200 was provided. As shown in FIG. 2, the microneedle mold 200 includes an upper surface 202 and molding holes 201 extending downward from the upper surface 202, wherein each of the molding holes 201 includes a tip and a bottom, the tip is away from the upper surface 202, and the bottom plane is flush with the upper surface 202.

In step (3), a stereo shape of the molding holes 201 in the microneedle mold 200 should be matched with the expected shape of the microneedles 110. As above, the molding holes can be in a shape of cylinder, cone, circular truncated cone, etc., or a combination of them, and preferably regular or irregular circular cone, conoid, triangular pyramid, rectangular pyramid or higher-level pyramid which may be a right cone or an oblique cone.

Each of the molding holes 201 should have a height, a bottom width and a tip spacing corresponding to those of the microneedles 110. However, in some cases, each of the molding holes 201 can further have a height and a bottom width larger than those of the microneedles 110. In the latter case, the formed microneedles 110 will not fill the whole space of the molding holes 201.

Further, the upper surface 202 (including inner surface of the molding holes 201) of the microneedle mold 200 was coated with an anti-bonding layer.

In the present invention, the microneedle mold 200 is commercially available; for example, it can be a customized PDMS mold purchased from Taizhou Chipscreen Medical Technology Company, and the mold parameters can be customized according to the needs of the needle body size. Specifically, in the microneedle mold 200 adopted in the example of the present invention, the heights of all molding holes 201 were 600 μm, the bottom widths were 320 μm, the tip spacings were 500 μm, and the overall microneedle mold 200 was L×W=15 mm×15 mm.

(4) The mixed solution obtained in step (2) was placed in the molding holes 201 and filled at least a portion of the volume of the molding holes 201.

In step (4), there is no special limitation to the volume of the molding holes 201 filled by the mixed solution, but preferably at least ¼, ⅓, ½, ⅔ and ¾ of the volume of the molding holes 201 is filled by the mixed solution, and most preferably the whole volume is filled by the mixed solution. When the whole volume of the molding holes 201 is not filled, the portion of the molding holes 201 that is not filled by the microneedles 110 should be filled by a backing material solution in step (6) below.

(5) The mixed solution containing the matrix material and the recombinant subunit lung-associated virus vaccine solution in the molding holes 201 was crosslinked and/or dried and cured to form microneedles 110 in the molding holes 201, and a plurality of microneedles 110 formed a recombinant subunit lung-associated virus vaccine microneedle array.

(6) A backing material-containing solution was applied to bottom surfaces of the microneedles 110 and the upper surface 202 of the microneedle mold 200 that was not covered by the microneedles 110 to form a backing solution layer, so that the recombinant subunit lung-associated virus vaccine microneedle array was attached to the backing solution layer.

In step (6), in the subunit vaccine microneedle product 100, the backing material is polyethylene glycol diacrylate, silk fibroin, GelMA, carboxymethyl cellulose, trehalose, hyaluronic acid, polylactic acid-glycolic acid copolymer, polylactic acid, galactose, PVP, polyvinyl alcohol or a mixture of two or more, and preferably hyaluronic acid.

(7) The backing solution layer obtained in step (6) and optional microneedle array simultaneously attached to the backing solution layer were dried and cured to form a subunit vaccine microneedle product 100.

In step (6) and step (7), a water content in the microneedles 110 and the backing 120 in the drying process is preferably lower than 20%, more preferably lower than 10%, and most preferably lower than 5%.

### Example 3

An application of the subunit vaccine microneedle product prepared in example 1 and the method for preparing the subunit vaccine microneedle product in example 2 in preparing medical devices and drugs against pneumonia.

In such example, the technical features and optimal ranges of the subunit vaccine microneedle product and the preparation method thereof are still applicable.

Test

Seventy healthy mice with an approximate body weight of 40 g were randomly divided into 10 groups with 7 mice in each group, and subjected to infection test through a control group and a test group, wherein five groups of mice in the test group were respectively applied with the microneedle vaccine prepared in example 4, and five groups of mice in the control group were respectively applied with blank microneedle product, which was a recombinant subunit preparation containing no microneedle vaccine of the present invention.

The chest hair of the mice was scraped, and the microneedle vaccine and blank microneedle product were respectively attached to the chest of the mice. After 7 d of vaccine attachment, the microneedle patches were removed, and the mice were infected with a mimicking virus. The EC50 of monoclonal antibody was tested by ELISA to determine the infection of the mice.

The lung-associated virus vaccine microneedle product and blank microneedle product were used for mouse test. The microneedle patches were applied to the skins of the points corresponding to the lungs of mice, and the mice were infected with the mimicking virus.

| | Group | | | | | | | | | |
|---|---|---|---|---|---|---|---|---|---|---|
| | Control Group | | | | | Test Group | | | | |
| Effect | 1 | 2 | 3 | 4 | 5 | 1 | 2 | 3 | 4 | 5 |
| IgG Log10 (ELISA EC50) | 0 | 0 | 0.9 | 0 | 0 | 1.4 | 1.4 | 1.4 | 2.3 | 1.5 |
| Body weight (g) of mice | 0 | 0 | 40 | 0 | 0 | 46 | 44 | 47 | 44 | 45 |
| Clinical score of mice | 0 | 0 | 6 | 0 | 0 | 7 | 7 | 6 | 7 | 7 |
| Survival result of mice | Dead | Dead | Alive | Dead | Dead | Alive | Alive | Alive | Alive | Alive |

| | | |
|---|---|---|
| Survival rate of mice | 20% | 100% |

Therefore, the present invention adopts the recombinant subunit vaccine microneedle product as well as the preparation method and the application thereof, wherein such microneedle product realizes a rapid immune response by loading a recombinant subunit lung-associated virus vaccine-containing solution in the microneedle product after being administered to the skin, and a long-acting stable release and rapid response of a lung-associated virus vaccine are achieved.

Finally, it should be stated that the above-mentioned embodiments are only used for describing, rather than limiting, the technical solutions of the present invention. Although the present invention is described in detail by reference to the preferred embodiments, those of ordinary skill in the art should understand that they can still make modifications or equivalent substitutions to the technical solutions of the present invention, but these modifications or equivalent substitutions will not make the modified technical solutions deviate from the spirit and scope of the technical solutions of the present invention.

1. A recombinant subunit vaccine microneedle product, comprising a backing and a recombinant subunit lung-associated virus vaccine solution-containing microneedle array attached to a side of the backing, wherein the recombinant subunit lung-associated virus vaccine solution-containing microneedle array comprises a plurality of microneedles, wherein each of the plurality of microneedles comprises a matrix and a recombinant subunit loaded in the matrix.

2. The recombinant subunit vaccine microneedle product of claim 1, wherein the recombinant subunit lung-associated virus vaccine is obtained by extracting a targeted protein structure of bacteria and viruses through a chemical decomposition or a controlled proteolysis method, and screening out a fragment lung-associated virus vaccine with immunocompetence.

3. The recombinant subunit vaccine microneedle product of claim 1, wherein the matrix is formed by crosslinking and/or drying and curing an aqueous solution containing at least one substance selected from the group consisting of: polyethylene glycol diacrylate, silk fibroin, methacrylate gelatin (GelMA), carboxymethyl cellulose, trehalose, hyaluronic acid, polylactic acid-glycolic acid copolymer, polylactic acid, galactose, polyvinylpyrrolidone (PVP), and polyvinyl alcohol, preferably GelMA and/or hyaluronic acid, and more preferably GelMA.

4. The recombinant subunit vaccine microneedle product of claim 1, wherein the backing is formed by crosslinking and/or drying and curing of an aqueous solution containing at least one substance selected from the group consisting of: polyethylene glycol diacrylate, silk fibroin, methacrylate gelatin (GelMA), carboxymethyl cellulose, trehalose, hyaluronic acid, polylactic acid-glycolic acid copolymer, polylactic acid, galactose, polyvinylpyrrolidone (PVP), and polyvinyl alcohol, and preferably hyaluronic acid.

5. The recombinant subunit vaccine microneedle product of claim 1, wherein each of the plurality of microneedles comprises a tip and a bottom, the tip is away from the backing, each of the plurality of microneedles is attached to the backing via the bottom, a distance from the tip to the bottom is 200 μm-1 mm, a diameter of the bottom is 100 μm-500 μm, and a spacing between the adjacent tips is 300 μm-800 μm.

6. A preparation method of the recombinant subunit vaccine microneedle product according to claim 1, comprising the following steps:
1) preparing a recombinant subunit lung-associated virus vaccine solution: extracting a targeted protein structure of bacteria and viruses through a chemical decomposition or a controlled proteolysis method, and preparing screened fragments with immunocompetence into the recombinant subunit lung-associated virus vaccine solution;
2) adding a matrix material forming the matrix to the recombinant subunit lung-associated virus vaccine solution obtained in step 1 to form a mixed solution; wherein in the mixed solution, the matrix material accounts for 20%-40% of a total weight of the mixed solution, preferably 25%-35%, and more preferably 30%;
3) preparing a microneedle mold;
4) placing the mixed solution obtained in step 2 in molding holes of the microneedle mold, and filling at least a portion of a volume of the molding holes, and preferably a whole volume of the molding holes;
5) crosslinking the mixed solution containing the matrix material and the recombinant subunit lung-associated virus vaccine solution in the molding holes, and performing ultraviolet crosslinking for 5 s-15 s, and preferably 10 s, and/or performing drying and curing, and preferably performing hot curing, to form the plurality of microneedles in the molding holes; wherein the plurality of microneedles form the recombinant subunit lung-associated virus vaccine microneedle array, and each of the plurality of microneedles comprises a tip and a bottom, and the tip is away from an upper surface relative to the bottom;
6) applying a solution containing a backing material to bottom surfaces of the plurality of microneedles and an upper surface of the microneedle mold that is not covered by the plurality of microneedles to form a backing solution layer, and crosslinking the backing material to form a continuous backing layer, so that the recombinant subunit lung-associated virus vaccine microneedle array is attached to the backing solution layer or the continuous backing layer; and
7) drying and curing the backing solution layer or the continuous backing layer obtained in step 6 and the recombinant subunit lung-associated virus vaccine microneedle array to form the recombinant subunit vaccine microneedle product.

7. The preparation method of claim 6, wherein the microneedle mold comprises the upper surface and molding holes extending downward from the upper surface, wherein each of the molding holes comprises a molding tip and a molding bottom, the molding tip is away from the upper surface, a plane of the molding bottom is flush with the upper surface, a distance from the molding tip to the molding bottom is 200 μm-1 mm, a diameter of the molding bottom is 100 μm-500 μm, and a spacing between the adjacent molding tips is 300 μm-800 μm.

8. An application of the recombinant subunit vaccine microneedle product according to claim 1 in preparing medical devices or drugs against pneumonia.
